(19) United States
(12) Patent Application Publication
(10) Pub. No.: US 2008/0171399 A1
(43) Pub. Date: Jul. 17, 2008

(54) Forensic Test for Human Blood

(75) Inventor: Karl Reich, Northfield, IL (US)

Correspondence Address: BAKER & MCKENZIE LLP, Pennzoil Place, South Tower, 711 Louisiana, Suite 3400, HOUSTON, TX, 77002-2716, US

(73) Assignee: INDEPENDENT FORENSICS, INC., Hillside, IL (US)

(21) Appl. No.: 11/869,747

(22) Filed: Oct. 9, 2007

Related U.S. Application Data
(60) Provisional application No. 60/828,693, filed on Oct. 9, 2006.

(51) Int. Cl. G01N 33/558; G01N 21/84

(52) U.S. Cl. 436/514; 422/55

(57) ABSTRACT

A lateral flow test strip for the definitive detection of human blood from forensic samples derived from crime scenes, sexual assault evidence kits, and other sources of forensic evidence.

### PRIOR RELATED APPLICATIONS

This application claims the benefit of U.S. Provisional Application Ser. No. 60/828,693 filed Oct. 9, 2006, entitled “Forensic Test for Human Blood,” which is incorporated herein in its entirety.

### FEDERALLY SPONSORED RESEARCH STATEMENT

Not applicable.

### REFERENCE TO MICROFICHE APPENDIX

Not applicable.

### FIELD OF THE INVENTION

This invention relates generally to the field of criminal and civil forensics and specifically to the definitive detection of human blood from forensic samples derived from crime scenes, sexual assault evidence kits, forensic evidence and sample derived from any red-brown stain on any fabric: carpet, clothing, bedding, or floor, wall, weapons, tools, and other relevant samples. This invention uses a lateral flow test with specificity for human blood which is previously undescribed. Current forensic methods to identify blood are not specific to human blood and exhibit high dose Hook effect. The described lateral flow test uses monoclonal antibodies configured as individual capture and detection components. This is the first such test available for the identification of human blood and is generally useful and novel for the forensic identification of blood from a wide variety of samples.

The detection of human blood is based on a specific, sensitive and unique assay for human glycophorin A, a major sialoglycoprotein in human erythrocytes. The suggested function of glycophorin A resides in the ability of the heavily glycosylated N-terminal extracellular domain to produce a mucin-like surface for erythrocytes to prevent aggregation. Although specifically designed for the detection of human blood from samples, swabs, exhibits, extracts and evidence derived from criminal and civil forensic investigations, the method can be used to detect human blood from any source or for any purpose.

In one embodiment, the invention involves the use of two monoclonal antibodies that each recognize a single non-overlapping epitope unique to human glycophorin A. The antibodies are configured in a lateral flow immunochromatographic strip test such that one antibody, the capture antibody, is immobilized on a membrane at a defined position. The second antibody, the detection antibody, which is labeled for visualization, is placed on the conjugate pad. Aqueous extracts to be tested for the presence of human blood are deposited onto the conjugate pad where a complex is formed consisting of labeled detection antibody and human glycophorin A present in the sample. The complex then migrates along the membrane and is captured by the immobilized capture antibody. This captured complex becomes visible to the naked eye as it is immobilized on the test line through binding to the capture antibody. The test is rapid, sensitive and specific for human glycophorin A and provides a definitive test for the presence of human blood.

### BRIEF DESCRIPTION OF THE DRAWINGS

FIG. 1: Lateral Flow Immunochromatographic Strip Test for Detection of Human Blood.

### DETAILED DESCRIPTION OF THE INVENTION

In one aspect, the present invention comprises a test for the presence of human blood that consists of two monoclonal antibodies which each have a single, unique non-overlapping epitope present on human glycophorin A.

Preferably, the monoclonal antibodies do not cross-react with the blood of domestic dog, domestic cat, cow, horse, chicken, pig, skunk, ferret, bonobo chimp, spider monkey, gorilla, Nubian goat, box turtle, American elk, orangutan, mule deer, Siberian tiger, alpaca, baboon (Canis familiaris, Felis domesticus, Bos taurus, Equus caballus, Sus domesticus, Mephitis mephitis, Mustela putorius furo, Pan Paniscus, Ateles geoffroyi, Gorilla gorilla, Capra ibex nubiana, Terrapene ornate, Cervus elaphus, Pongo pygmaeus, Odocoileus hemionis, Panthera tigris altaica, Lama pacos, Papio cynocephalus respectively).

In a preferred embodiment, one of the monoclonal antibodies is determined to be the ‘capture’ antibody by immunologically based testing with authentic human glycophorin A. Similarly, a second monoclonal antibody is determined to be the ‘detection’ antibody by immunologically based testing with authentic human glycophorin A.

In another aspect, the invention is directed to a lateral flow test strip wherein one of the monoclonal antibodies, the capture antibody, is immobilized on a solid substrate, bead or surface and used with the detection antibody, which is labeled or derivatized and the pair used to detect human glycophorin A.

In another embodiment, the invention is directed to monoclonal antibody BRIC 163 for the detection antibody, and monoclonal antibody BRIC 256 for the capture antibody.

In another embodiment, the invention is directed to monoclonal antibody BRIC 256 for the detection antibody, and monoclonal antibody BRIC 163 for the capture antibody.

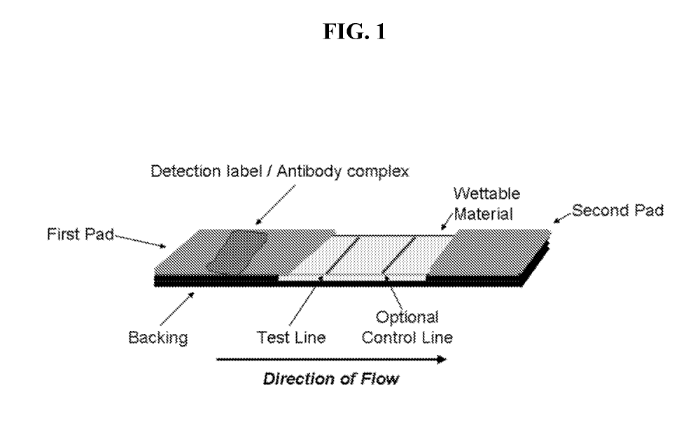

FIG. 1 depicts the design of the lateral flow test using antibodies specifically directed and specific to human glycophorin A.

As shown in FIG. 1, the three components of the lateral flow test are the conjugate pad, the membrane, and the wick (sometimes referred to herein as the absorbent pad).

The conjugate pad can be made from absorbent material such as glass-fiber paper, rayon, cotton or polyester.

The membrane can be made from nitrocellulose with or without a non-absorbent backing.

The wick can be made from absorbent material such as glass-fiber paper, rayon, cotton or polyester.

An additional component, a sample pad, is sometimes used in addition to the conjugate pad and functions as secondary filter for complex extracts and is placed above the conjugate pad and can be made from absorbent material such as glass-fiber paper, rayon, cotton or polyester.

The conjugate pad contains antibodies labeled for visualization (colloidal gold, latex beads, fluorescent compounds, enzymes, biotin or other reagents can be used for final visualization or visual enhancement) that are specific for human glycophorin A molecules. The membrane has been configured to contain a ‘control line’ and a ‘test line’. The test line contains glycophorin A antibody molecules responsible for immobilizing the glycophorin A-gold conjugate or similar complexes as the sample passes over the membrane; the test line has the capture antibody that may be a monoclonal antibody. The control line contains an anti-antibody source (anti-mouse, anti-rabbit, anti-goat or anti-sheep or anti-horse) Ig antibody and fixes non-bound gold-conjugate antibodies (or similarly labeled antibodies ) indicating that the tested sample has indeed immunochromatographed past the test line. The wick soaks up the sample to prevent backflow during the detection time window. Electro-mechanical dispensers are used to dispense the colloidal gold-glycophorin A antibody complexes (or similarly labeled antibodies) onto the conjugate pad and the test and control lines on the immunochromatographic membrane.

In use, a designated volume from an aqueous extraction of a forensic sample is placed at the conjugate pad end of the test strip and capillary action directs the sample across the conjugate pad. As the sample moves across the conjugate pad, the conjugate is redissolved in the aqueous liquid front as it passes through the pad and any glycophorin A molecules present in the sample (if the sample contains glycophorin A) are specifically bound by the gold conjugate complexed with the anti-glycophorin A antibody. As the sample continues across the membrane, the anti-glycophorin A antibody molecules dispensed at the immobilized test line bind the glycophorin A-labeled antibody complex.

The red test line on FIG. 1 illustrates a positive reaction for human glycophorin A. The control line indicates the glycophorin A-conjugate complex has been properly released from the conjugate pad and has immunochromatographed past the test line, confirming the proper functioning of the immunochromatographic strip.

### EXAMPLE 1

### PREPARATION OF MONOCLONAL ANTIBODIES

Immunization: BALB/C mice are selected and immunized with the protein fraction of seminal plasma containing glycodelin-S and other proteins in Freud's adjuvant. A two to three week schedule of immunization and boosters is used and responder animals are identified by tail vein bleeds and analysis by ELISA. Positive responder animals receive one additional boost and are prepared for splenic fusion.

Spleen cells, isolated from sacrificed animals are fused with previously prepared myeloma cells. Cell fusion is carried out by co-centrifugation of freshly harvested spleen cells with myeloma cells in polyethylene glycol, and subsequent distribution into 96 well plates with or without feeder layers.

Culture supernatants from surviving fusions, i.e., individual wells, are tested for mouse IgG production and for production of IgG that recognizes antigen - testing is performed by ELISA or similar assay. Myeloma fusion cells from Ig producing antigen positive wells are cloned by limiting dilution, grown and retested for Ig production and for specificity to the immunization antigen before being expanded. Cloning by limiting dilution can be performed twice to insure that antibody producing cells are true clones. Generally four to six frozen stocks of each Ig positive clone are prepared and stored in liquid nitrogen for each cloned Ig positive well. Antibodies from selected wells are then tested in suitable and various combinations to determine if they recognize non-overlapping epitopes on the immunizing antigen.

### EXAMPLE 2

### COLLOIDAL GOLD LABELING OF ANTIBODIES

Colloidal gold solution is monitored by optical absorbance at 521-525 nm. For 2 L of colloidal gold solution take 24 ml of 1.14% gold chloride and 40 ml of 1.14% sodium citrate in a baffled 4 L flask added to 18.2 mega ohms purity water.

1. Boil 2.28 L H₂O until to a rolling boil; trap water vapor to prevent loss of volume (can use an inverted 125 ml flask or a large evaporating dish).

2. Start timer: at 30 seconds add 24 ml 1.14% gold chloride making addition along side of flask; increase stirring from slow to medium.

3. At exactly 2 minutes, add 40 ml 1.14% sodium citrate directly into middle of stirring solution.

4. Continue stirring for an additional three minutes for a total of 5 minutes.

The solution will undergo various color changes. Upon addition of the gold chloride, the solution turns yellowish; upon addition of the sodium citrate, the solution goes from yellow to clear (at about 2 min 30 sec). At about 3 minutes, the solution turns completely black, then turns purplish, and then at 4 minutes it will appear reddish-purple. At 5 minutes, the solution is a deep cherry red.

After 5 minutes, when the solution is cherry red, put flask in water bath to cool down.

Note: For gold chloride solution use Teflon coated spatula (as Au₃Cl will corrode metal spatulas). Make 1.14% solution before use: 1.14 g of Gold (III) chloride trihydrate (G4022, Sigma—stored in refrigerator, desiccated and protected from light) in 100 ml H₂O. Filter solution through 0.2 micrometer filter. Keep solution refrigerated and protected from light (foil covered, amber bottle).

Sodium citrate is prepared as an aqueous solution and filtered before use (0.2 micrometer filter).

Optical density of gold colloidal solution is scanned from 400-600 nm and the peak and wavelength maxima noted. Typical values are peak @525 nm, O.D.=1.109.

Conjugation of colloidal gold particles to antibodies is empirically determined for each batch of antibodies and is sensitive to the pH and pl of the antibodies.

Use 0.2 M potassium carbonate to adjust pH of colloidal gold solution: pH of starting solution will be 4.0. Adjust trial solutions of 7.5 ml to pH of 7.0, 7.8, 8.2, 8.6, and 9.0 (do not add acid to balance pH, add more colloidal gold solution).

Mix with antibody (prepared at 0.2 mg/ml and diluted into 2 mM phosphate buffer, pH 9.0), and test 0, 6 μg/ml, 8 μg/ml, 10 μg/ml, and 12 μg/ml solutions of antibody with the above pH ranges of colloidal gold.

| Antibody | | Ab (ml) | | Buffer (ml) | Gold | NaCl |
|---|---|---|---|---|---|---|
| 0 | | 0 | | 50 ml | 500 | 100 |
| 6 | μg/ml | 16.5 | ml | 33.5 ml | 500 | 100 |
| 8 | μg/ml | 22 | ml | 28 ml | 500 | 100 |
| 10 | μg/ml | 27.5 | ml | 22.5 ml | 500 | 100 |
| 12 | μg/ml | 33 | ml | 17 ml | 500 | 100 |
| 16 | μg/ml | 44 | ml | 6 ml | 500 | 100 |

Mix antibody and colloidal gold solutions as shown (see table) and allow to stand at room temperature for 2 minutes.

Add NaCl (10% w/v in H₂O, pre filtered through 0.22 μm).

Wait 5 minutes and read the OD of each solution and determine λ max.

Optimal concentration of antibody is determined by observing the color of the antibody-gold mixture upon addition of salt. The appearance of a transparent cherry-red solution indicates an optimal concentration of antibody where no unbound gold particles exist. The appearance of a cloudy purple or black solution indicates precipitation of unbound gold particles has occurred and a non-optimal concentration of antibody.

### EXAMPLE 3

### CONJUGATION OF COLLOIDAL GOLD AND GLYCOPHORIN A ANTIBODY

The antibodies are obtained from University of Bristol, Bristol Institute for Transfusion Sciences incorporating the International Blood Group Reference Laboratory, IBGRL Research Products, National Blood Service, Southmead Road, Bristol, BS10 5ND, England, United Kingdom. MAb “BRIC 256” (Batch 2038L) Product Code 9415 P; MAb “BRIC 163” (Batch 695L) Product Code 9410P.

For conjugation of 20 ml colloidal gold with BRIC 256 antibody, 200 micrograms BRIC 256 antibody (for 10 μg/ml concentration) is added to 20 ml colloidal gold, pH 8.2, and gently rocked for 30 minutes after which 2.2 ml of 10% BSA (for 1% BSA final concentration) is added and gently rocked for 1 hour.

For conjugation of 20 ml colloidal gold with BRIC 163 antibody, 200 micrograms BRIC 163 antibody (for 10 μg/ml concentration) is added to 20 ml colloidal gold, pH 9.0, and gently rocked for 30 minutes after which 2.2 ml 10% BSA (for 1% BSA final concentration) is added and gently rocked for 1 hour.

### EXAMPLE 4

### CONCENTRATION OF COLLOIDAL GOLD CONJUGATE

BRIC 256 and BRIC 163 gold conjugate is spun down at 16,000 G for 25 minutes with no brake at 4 degrees. Supernatant is carefully removed and 1.8 ml of passive gold diluent (0.07% sodium phosphate and 0.1% BSA) is added for an approximate 10-fold dilution. The OD of the colloidal gold conjugate needs to be 10, therefore, the volume of conjugate is adjusted (if necessary) to obtain optimal OD after monitoring absorbance at 523 nm.

### EXAMPLE 5

### PRETREATMENT OF CONJUGATE PAD

Glass fiber conjugate pad material is cut into strips 300 mm long by 22 mm wide. To make the conjugate pad more hydrophilic, the strips are pretreated by immersion in a solution containing sodium tetraborate (0.2%), Bovine Serum Albumin (BSA, 3%), polyvinylpyrrolidone (PVP, 1%), sucrose (0.1%), and Triton X-100 (0.25%) for 10 minutes (5 minutes on each side). The treated pads are blotted dry and dried in an oven at 37° C. for one hour. The dried conjugate pads are stored in an airtight and moisture resistant foil pouch containing desiccant.

### EXAMPLE 6

### DISPENSING OF COLLOIDAL GOLD CONJUGATES ONTO TREATED CONJUGATE PADS

Twenty percent (20%) w/v sucrose and 5% w/v trehalose is added to 1 ml BRIC 256- and BRIC 163-colloidal gold conjugate. BRIC 256 and BRIC 163 conjugate are each dispensed onto a pretreated conjugate pad at 10 μl/cm using an AIRJET QUANTI™ dispenser (BIODOT® Inc., Irvine, Calif.). After dispensing the conjugates onto the pads, the conjugate pads are dried at 37 degrees for 1 hour, and then stored with desiccant.

### EXAMPLE 7

### DISPENSING OF BRIC 256 AND BRIC 163 AND GOAT ANTI-MOUSE IGG ONTO NITROCELLULOSE MEMBRANES

BRIC 256 and BRIC 163 are dispensed onto a nitrocellulose membrane using a BIODOT QUANTI™ dispenser (BioDOT®) at 1 μl/cm and 0.8 mg/ml antibody solution (called the test line). Goat anti-mouse IgG antibody is dispensed onto the same membrane as BRIC 256 and BRIC 163, at a location 4 mm above the test line (called the control line). The membranes are dried at 37 degrees for 30 minutes.

### EXAMPLE 8

### LATERAL FLOW STRIP TEST MATERIALS

A variety of test materials must be used to determine the optimal combination of membrane, conjugate pad and blocking solutions with each antibody combination. These include different nitrocellulose membranes, different conjugate pad materials, different mixtures of detergents and buffers for blocking. The use of sample pads must also be tested depending of the sample origin.

### EXAMPLE 9

### ASSEMBLY OF LATERAL FLOW STRIP TEST

The 300 mm backing cards are assembled by removing the adhesive tape and placing the membrane in the middle, the wick at the top, and the conjugate pad on the bottom, as described below. The backing cards are assembled with BRIC 256 gold conjugate/BRIC 163 test line or BRIC 163 gold conjugate/BRIC 256 test line. A lamination instrument that holds the individual components of the assay with mild vacuum and then seals the components together. The Laminator has a lid and a base, both of which use vacuum to hold components in the correct orientation. The lid holds the backing card, with adhesive facing outward and the base holds the components of the strip test in pre-machined grooves. Components are placed on the base in the following order (Note: components are placed in reverse order taking into account the geometry of the laminator):

1. Place the sample pad in the top groove in base near the hinge.

2. Place the conjugate pad in the next slot, overlapping the sample pad.

3. Place the membrane in the middle slot, with the edges of the membrane overlapping the wick and the conjugate pad by ˜2 mm. Lower the top of the laminator and press down to adhere the components together. Turn off vacuums feeds for both lid and base and raise the lid.

Assembled and laminated strips are cut into 3-5 mm sections using a dedicated step motor driven guillotine cutter. The backing card consisting of the assembled and pressed components are fed into the device between the material guide rails (rails are adjusted for the width of the assembled strip). The backing cards (on which the assembled strip test components are adhered) are advanced until the front edge of the card reaches the (closed) blade. Cutting is programmed and begins when the hand-held controller is set to ‘go’ (cutting will not proceed if the Plexiglas cover is not pressed closed due to interlock switches). Assembled immunochromatographic wick, membranes, backing and pads are cut into strips and stored with desiccant until use. The cut strip tests (FIG. 1) can then be placed into appropriate cassettes or used directly. If used directly, single strips are placed in glass culture tubes containing ˜100 μl of the specified test solution with the conjugate side of the strip test direct toward the bottom of the glass tube. The test solution will then diffuse up the strip toward the wick. The reaction can be stopped by removing the strip from the glass culture tubes, laying the strip on a paper towel, and removing the conjugate pad with forceps.

What is claimed is:

1. A method of detecting human blood in a sample by contacting an aqueous aliquot of said sample with an anti-glycophorin A antibody on a lateral flow test strip.

2. A lateral flow test strip comprising a detection antibody comprising a labeled anti-glycophorin A antibody, and a capture antibody comprising an anti-glycophorin A antibody fixed to said lateral flow test strip.
